(19) United States
(12) Patent Application Publication
(10) Pub. No.: US 2004/0059343 A1
(43) Pub. Date: Mar. 25, 2004

(54) Novel enhanced system for intraocular lens insertion

(76) Inventors: Kevin Shearer, Seattle, WA (US); Mark Cole, Trabuco Canyon, CA (US); Robert Ziegler, Meridian, ID (US)

Correspondence Address: STRADLING YOCCO CARLSON & RAUTH, SUITE 1600, 660 NEWPORT CENTER DRIVE, P.O. BOX 7680, NEWPORT BEACH, CA, 92660, US

(21) Appl. No.: 10/412,431

(22) Filed: Apr. 11, 2003

Related U.S. Application Data
(60) Provisional application No. 60/413,512, filed on Sep. 25, 2002.

(51) Int. Cl. A61F 9/00

(52) U.S. Cl. 606/107

(57) ABSTRACT

An apparatus for controllably delivering an IOL into the eye of a patient is disclosed. The device is configured for convenient and reliable positioning of a plunger rod assembly at different stages of the IOL injection procedure, such that various aspects of the insertion procedure can be performed precisely and reliably by a user of the device. In addition, the present invention assists the user during the procedure by accurately and consistently positioning the plunger rod assembly prior to ejection of the folded or rolled IOL from the insertion device.

### CROSS REFERENCE TO RELATED APPLICATIONS

The present application claims priority to U.S. Provisional Patent Application No. 60/______, filed Sep. 25, 2002, entitled “SYSTEM FOR IOL INSERTION” the contents of which are fully incorporated herein by reference.

### BACKGROUND OF THE INVENTION

When a natural lens of the eye becomes occluded, it is conventional practice to remove the occluded lens, such as by phacoemulsification, and replace the natural lens with an artificial intraocular lens (IOL). During cataract surgery, a small incision is made in the cornea and the IOL is implanted in the eye as a replacement for the natural crystalline lens after cataract surgery. Alternatively, an IOL (e.g., phakic IOL) may also be used to alter the optical properties of or provide vision correction to an eye in which the natural lens remains.

An IOL often includes an optic and, preferably, at least one flexible fixation member or haptic, which extends from the optic and becomes affixed in the eye to secure the lens in position. The optic of the IOL normally includes an optically clear lens. As described above, implantation of an IOL into the eye involves making an incision in the eye. Preferably, the incision is made as small as possible to reduce trauma, speed healing and decrease patient recovery time.

In order to decrease the size of the corneal incision required for insertion of the IOL, the IOL can be formed of resilient material that can be “folded” or rolled for insertion into the lens capsule. The foldable (deformable) IOL automatically unfolds after it has passed through the incision. A substantial number of instruments have been devised to aid in inserting such a foldable lens into the eye. The advantages of the foldable lens in cataract removal and lens replacement are so significant that the majority of lens replacement procedures are performed with folded lenses inserted into the eye and released therein to assume their initial unfolded state.

Conventional insertion devices employ a hollow insertion tube having a diameter that permits the folded IOL to pass through the hollow space defined by the tube without permanent deformation. These devices typically include a plunger assembly having a rod, often made of metal, which is moved longitudinally in the hollow space of the device and in contact with the optic of the IOL to push the IOL through the hollow space.

Several disadvantages are apparent in such insertion devices. For example, pushing, without trapping or holding, the IOL through and out of the hollow space defined by the tube can cause the IOL to be released from the insertion device without precise control. The uncontrolled release of the IOL may damage the eye and/or cause the IOL to be incorrectly positioned within the capsule of the eye.

Other disadvantages of conventional insertion devices involve the plunger assembly. For example, the metal rod of the insertion device can mark the surface of the optic and/or even tear the optic, particularly when the optic is made of soft materials, such as soft elastomeric silicone polymeric materials. In addition, the metal rod may completely by-pass the IOL in the hollow space. That is, as the rod is being moved distally through the hollow space of the device, the rod may actually pass through a fold in the folded optic. If this occurs, the rod becomes ineffective to push the IOL through the hollow space. This problem has been avoided in the past by increasing the cross-sectional area of the rod. However, a rod with a large cross-sectional area presents its own problems. For example, pushing such a large rod through the hollow space can damage the IOL and/or the inserter or result in an uncontrolled release of the IOL into the eye, possibly with inserter debris being disadvantageously introduced into the eye.

In view of the foregoing, it is apparent that there is a need for an IOL insertion device that can fold and controllably deliver an IOL into the eye of a patient. In addition, the device should be configured so that the IOL can be inserted through an optimally small incision in the cornea of the eye. There is also a need to provide a method of using such a device that is uncomplicated, efficient and cost effective. Such a device and method should further include properties that reduce the incidence of tissue trauma over a variety of material designs and properties, facilitate proper IOL delivery and improve patient recovery times.

### BRIEF SUMMARY OF THE INVENTION

The present invention provides an IOL insertion device that can fold and controllably deliver an IOL into the eye of a patient. The device is configured for convenient and reliable positioning of the plunger rod assembly at different stages of the IOL injection procedure, such that various aspects of the insertion procedure can be performed precisely and reliably by a skilled technician rather than the surgeon. In addition, the present invention assists the surgeon during the procedure by accurately and consistently positioning the plunger rod assembly prior to ejection of the folded or rolled IOL from the insertion instrument.

It is an object of the present invention to provide an IOL insertion device configured to allow insertion of the IOL through an optimally small incision in the cornea of the eye.

It is a further object of the present invention to provide an IOL insertion device that accommodates a variety of IOL materials including, but not limited to, silicone and hydrophobic acrylics.

It is a further object of the present invention to provide an apparatus and methods of use wherein the desired insertion of the IOL is easily, controllably and effectively achieved over a variety of material factors.

It is a further object of the present invention to provide a method of using an IOL delivery device that is uncomplicated, efficient and cost effective.

It is a further object of the present invention to provide a device and method that reduce the incidence of tissue trauma, facilitate proper IOL delivery and improve patient recovery times.

It is a further object of the present invention to provide a spring-biased device that provides a user of the device with correct or sufficient pressure when using the insertion device of the present invention. In this regard, the device creates a continuous back pressure that allows the user to controllably release the IOL into the patient's eye when the IOL begins to exit the cartridge. As such, even with reduced material/cartridge friction as the IOL exits the cartridge, the potential to overshoot or launch the IOL in the eye too quickly is reduced and/or eliminated. In addition, the device allows the user to apply pressure consistently to the IOL as it moves along the shaft of the device, thereby reducing or eliminating any sudden starts and stops during the injection procedure.

These and other objects not specifically enumerated here are addressed by the present invention which, in at least one embodiment, may include a device for inserting, controllably releasing and accurately positioning a folded intraocular lens into an eye comprising a plunger rod assembly in communication with an inserter housing, wherein the inserter housing is adapted to house an intraocular lens. The device also includes a first drive mechanism for providing contact between and causing lateral movement of the plunger rod assembly and the intraocular lens within the inserter housing. The device may further include a positioning mechanism for preventing proximal lateral movement of the plunger rod assembly so that the intraocular lens may be accurately positioned within the eye.

The present invention also contemplates a method of inserting an intraocular lens into an eye. The method includes providing an insertion device comprising a plunger rod assembly, an inserter housing and a control knob assembly. The method also includes providing a cartridge having a folded intraocular lens positioned therein, loading the cartridge into the inserter housing, actuating the control knob assembly to couple a distal end of the plunger rod assembly with the intraocular lens and inserting a distal end of the cartridge into the eye. The method further includes applying a force to the control knob assembly to move the intraocular lens through the cartridge and partially eject the intraocular lens from the cartridge, actuating a latch and pin mechanism of the plunger rod assembly and removing the force to the control knob assembly without causing movement of the plunger rod assembly, positioning the intraocular lens in the eye, applying a force to the control knob assembly to eject the intraocular lens into the eye, and removing the distal end of the cartridge from the eye.

The present invention further contemplates an intraocular lens insertion device comprising an inserter housing comprising a plunger rod assembly and a control knob assembly, the control knob assembly having an unlocked position and a locked position the device also includes a cartridge for housing an intraocular lens, the cartridge adapted to be received within the inserter housing and engageable with the plunger rod assembly, a drive mechanism in communication with the control knob assembly and the plunger rod assembly and capable of causing lateral movement of the plunger rod assembly via the control knob assembly. The device further includes a biasing mechanism in communication with the plunger rod assembly to limit the lateral movement of the plunger rod assembly, and a locking mechanism in communication with the control knob assembly for preventing lateral movement of the plunger rod assembly when the control knob assembly is in the locked position.

### BRIEF DESCRIPTION OF THE DRAWINGS

Other features and advantages of the present invention will be seen as the following description of particular embodiments progresses in conjunction with the drawings, in which:

FIG. 1 is perspective view of an embodiment of an insertion device in accordance with the present invention;

FIG. 2 is a sectional view of an embodiment of an insertion device in accordance with the present invention;

FIG. 3 is an exploded view of an embodiment of an insertion device in accordance with the present invention;

FIG. 4 is a perspective view of an embodiment of an intraocular lens holding cartridge in accordance with the present invention;

FIG. 5 is a perspective view of an embodiment of a pushing member in accordance with the present invention;

FIG. 6 is a side perspective view of an embodiment of a pushing member in accordance with the present invention;

FIG. 7 is an end view of an embodiment of a pushing member in accordance with the present invention;

FIG. 8 is a perspective view of an embodiment of an elongate body in accordance with the present invention;

FIG. 9 is a side perspective view of an embodiment of an elongate body in accordance with the present invention;

FIG. 10 is a sectional view of an embodiment of an elongate body in accordance with the present invention;

FIG. 11 is an end view of an embodiment of an elongate body in accordance with the present invention;

FIG. 12 is a sectional view of a distal end of an embodiment of an elongate body in accordance with the present invention;

FIG. 13 is a perspective view of an embodiment of a latch pin in accordance with the present invention;

FIG. 14 is an end view of an embodiment of a latch pin in accordance with the present invention;

FIG. 15 is a side view of an embodiment of a latch pin in accordance with the present invention;

FIG. 16 is a perspective view of an embodiment of a control knob assembly in accordance with the present invention;

FIG. 17 is an end view of an embodiment of a control knob assembly in accordance with the present invention;

FIG. 18 is a sectional view of an embodiment of a control knob assembly in accordance with the present invention;

FIG. 19 is a perspective view of an embodiment of a forward tube in accordance with the present invention;

FIG. 20 is a top sectional view of an embodiment of a forward tube in accordance with the present invention;

FIG. 21 is a side sectional view of an embodiment of a forward tube in accordance with the present invention;

FIG. 22 is an end view of an embodiment of a forward tube in accordance with the present invention;

FIG. 23 is another end view of an embodiment of a forward tube in accordance with the present invention;

FIG. 24 is a perspective view of an embodiment of a clip in accordance with the present invention;

FIG. 25 is a top sectional view of an embodiment of a clip in accordance with, the present invention;

FIG. 26 is a side sectional view of an embodiment of a clip in accordance with the present invention;

FIG. 27 is a sectional view of a distal end of an embodiment of a clip in accordance with the present invention;

FIG. 28 is an end view of an embodiment of a clip in accordance with the present invention;

FIGS. 29-33 illustrate of a method of operating an embodiment of an insertion device in accordance with the present invention; and

FIGS. 34-37 illustrate alternate embodiments of latching mechanisms in accordance with the present invention.

### DETAILED DESCRIPTION OF THE INVENTION

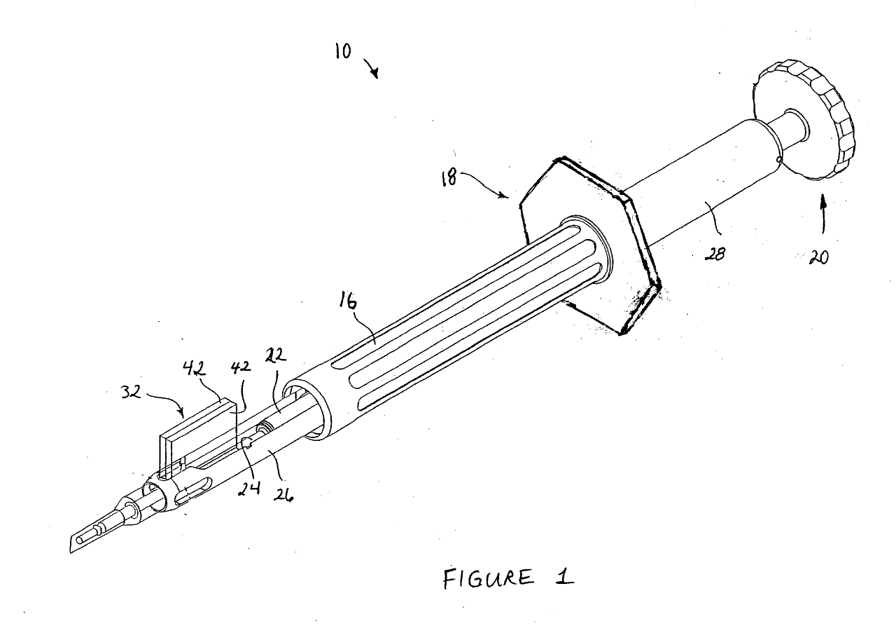

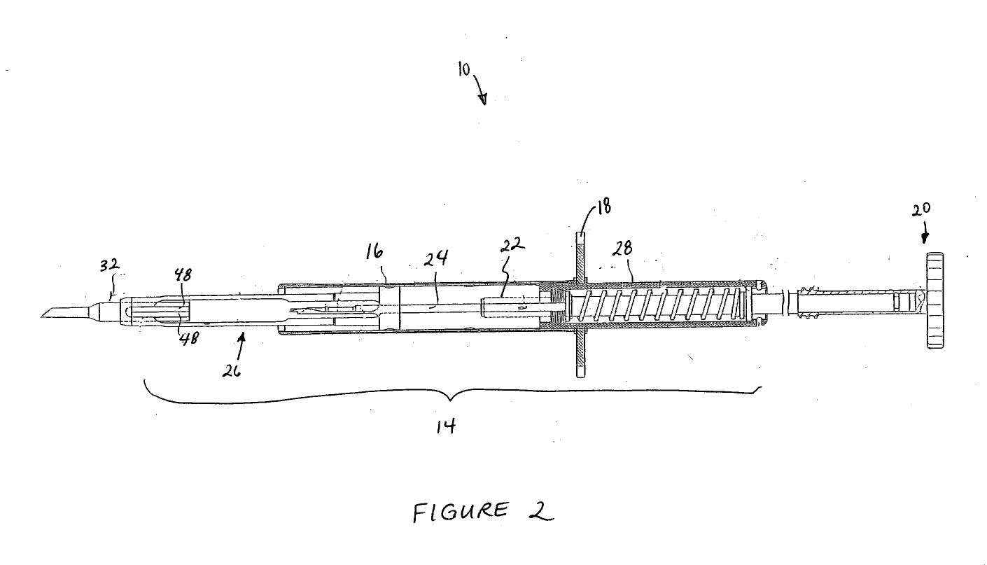

Referring to FIGS. 1-3, an embodiment of an IOL insertion device 10 in accordance with the present invention includes a plunger rod assembly 12, an inserter housing 14, a protective sheath 16, a finger grip 18 and a control knob assembly 20. The plunger rod assembly 12 includes an elongate body 22 and a pushing member 24 that act to controllably push the IOL out of the cartridge and into the lens capsule (not shown). The inserter housing 14, which includes a forward tube 26 and a rearward tube 28, houses the plunger rod assembly 12 and includes a chamber 30 configured to hold the cartridge (not shown) during an insertion procedure. Surrounding the inserter housing 14 are a protective sheath 16 and finger grip 18. The finger grip 18 and/or control knob (or handle) assembly 20 allow a user of the device 10 to accurately and consistently position the plunger rod assembly 12 for controlled release of the IOL into the chamber of the eye, as explained in further detail below.

The inserter device 10 of the present invention is used in combination with a lens holding cartridge 32. Lens holding cartridges 32, such as those disclosed in U.S. Pat. No. 6,398,788 and U.S. Pat. No. 6,334,862, whose contents are fully incorporated herein by reference, generally include a lens-folding portion 34, a holding chamber 36 and a hollow injection tube 38. As shown in FIG. 4, the lens-folding portion 34 includes a loading compartment 40 and integral hinged tabs 42 movable to open and closed states. The open state permits placement of the IOL (not shown) into the lens-folding portion of the cartridge 32, whereas the closed state folds the IOL and holds it in the resulting cylindrical lumen. During the insertion procedure, explained in further detail below, the folded IOL is advanced through the holding chamber 36 and into the hollow injection tube 38 of the cartridge 32. The decreased diameter of the injection tube 38, further folds and compresses the IOL so that the IOL can be passed through an incision in the eye no larger than about 4 mm.

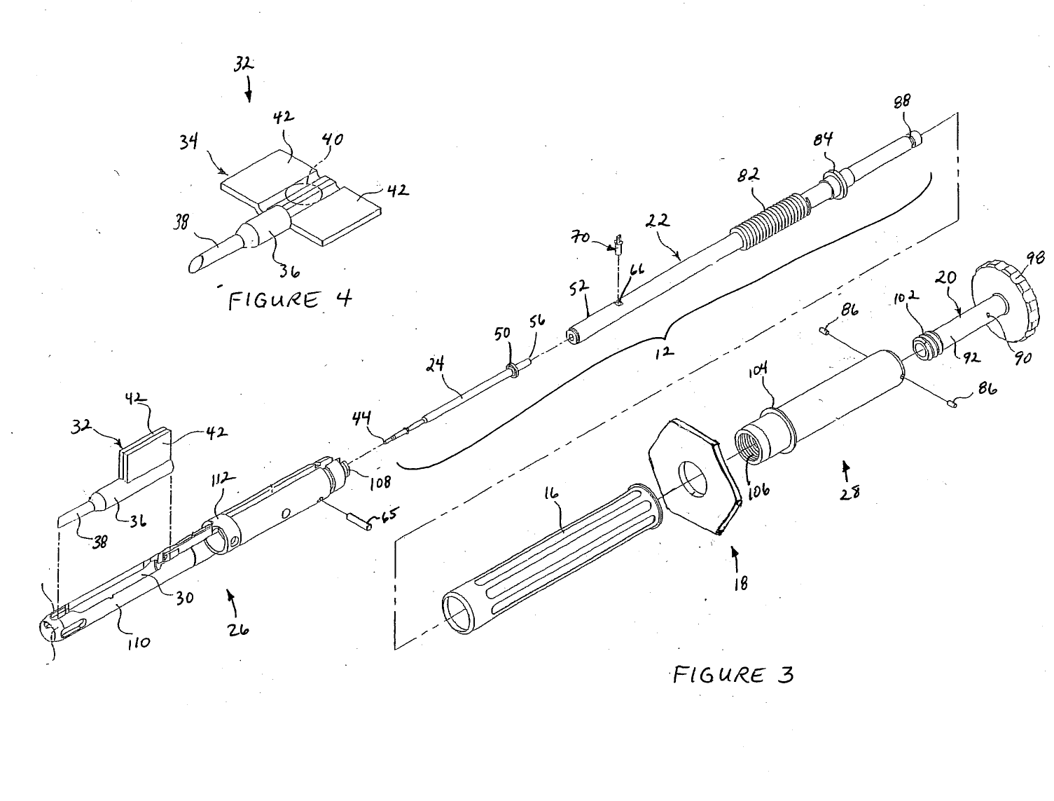

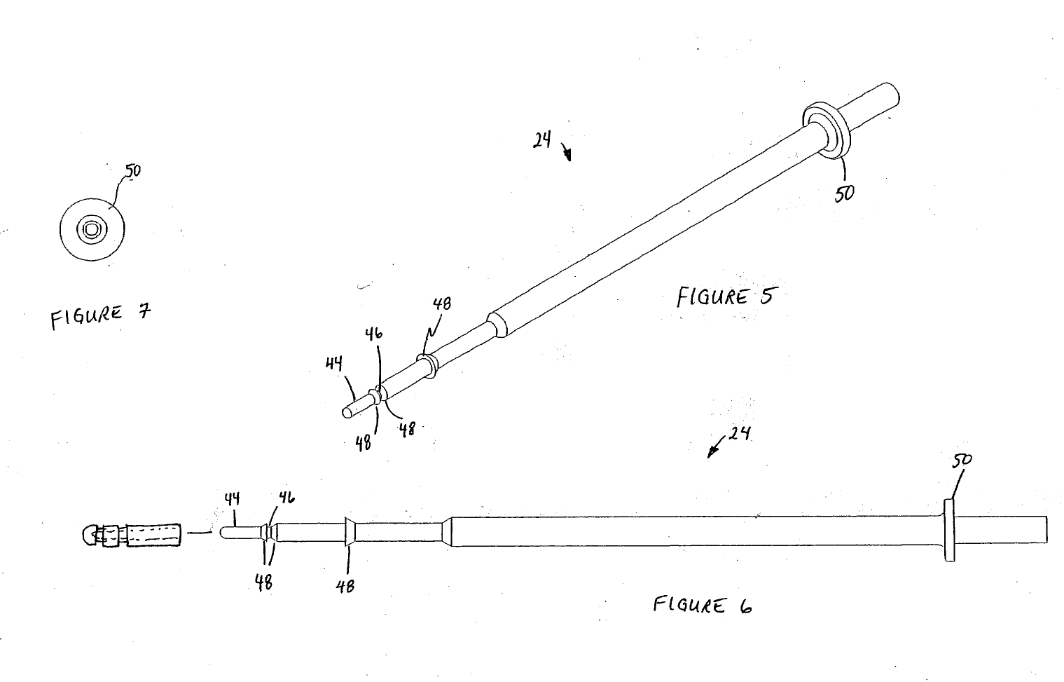

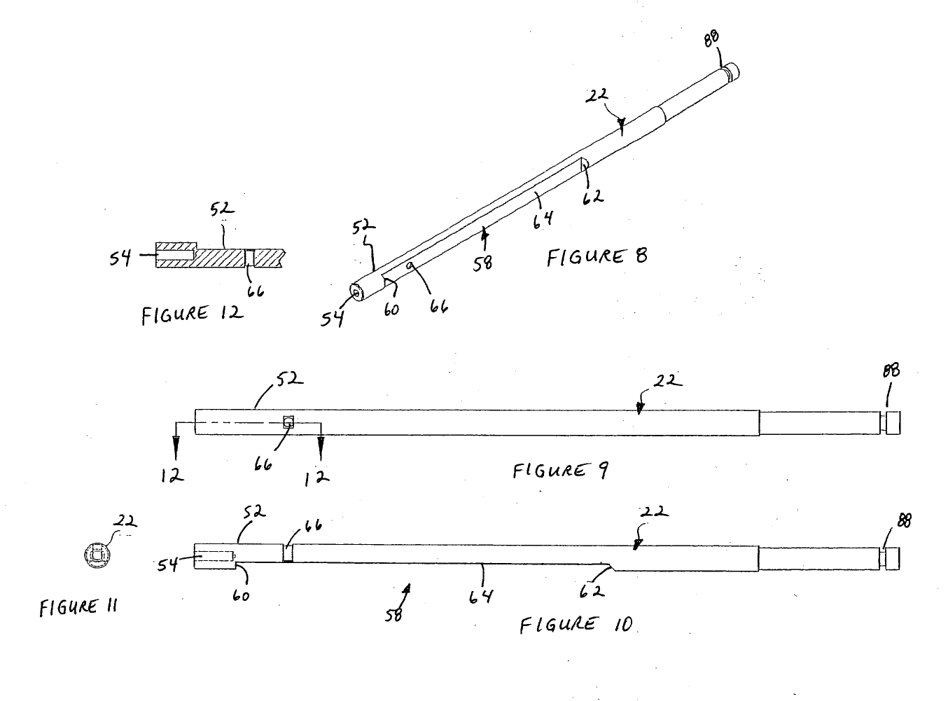

As noted in the Background of the Invention set forth above, uncontrolled release or ejection of the IOL from the insertion device 10 may damage the eye and/or cause the IOL to be incorrectly positioned within the capsule of the eye. As the present invention substantially eliminates these undesirable characteristics, it is instructive to describe the components of the device 10 that allow for convenient, reliable and controlled delivery of the IOL during an insertion procedure. For this purpose, reference is made to FIGS. 3 and 5-15.

Plunger Rod Assembly

FIGS. 3 and 5-15 illustrate an embodiment of the plunger rod assembly 12 of the present invention. The plunger rod assembly 12 may be fabricated as a disposable, single-use component or a reusable, multi-use component. As such, a variety of materials may be used to fabricate the plunger assembly 12. These materials include, but are not limited to, metals (such as stainless steel, aluminum or titanium), ceramics, plastics and the like, including combinations thereof. By way of illustrative example, with no limitation being intended or implied, the reusable plunger rod assembly 12 is fabricated from stainless steel to enable autoclaving after each use.

As shown in FIGS. 5-7, the cylindrically shaped pushing member 24 of the plunger rod assembly 12 includes a blunt or rounded distal tip 44 that contacts and/or pushes the IOL through and out of the cartridge 32 during an insertion procedure. In one embodiment of the invention, the distal tip 44 is fabricated from a relatively soft material, such as silicone polymeric elastomer. In an alternate embodiment, the tip 44 is fabricated from a rigid material and surrounded by a soft, elastomeric coating or cover. Preferably, the distal tip 44 of the pushing member 24 is fabricated from one or more materials and designed to prevent marks or tears in the IOL during the insertion procedure.

Proximal to the distal tip 44 is an annular channel 46 and several annular chamfered edges 48. The channel 46 and chamfered edges 48 provide a friction fit to secure the soft tip 44 onto the pushing member 24 of the insertion device 10. The resulting ridges and grooves of the soft tip 44, together with additional channels and chamfers formed on the exterior surface of the soft tip 44, function to hold the soft tip 44 in the fold of the IOL during an insertion procedure. In particular, as the tip 44 passes through a bore in the cartridge 32 and contacts the IOL, the surface configuration of the tip 44 provides additional contacts points with the IOL which facilitate in pushing the IOL through the cartridge 32.

Proximal to the channel 46 of the pushing member 24 is an annular rib or projection 50. The projection 50 functions as a shoulder or stop to properly locate and position the pushing member 24 within the elongate body 22.

FIGS. 8-14 illustrate the cylindrically shaped elongate body 22 of the plunger rod assembly 12 in accordance with one embodiment of the present invention. As shown in FIGS. 8-12, the distal end 52 of the elongate body 22 includes an axial, interior bore 54 that extends along a portion of the elongate body 22 and is sized to accommodate the proximal end 56 of the pushing member 24. To assemble the two components 22, 24, the proximal end 56 of the pushing member 24 is simply press-fit into the distal bore 54 of the elongate body 22. Although the illustrated embodiment of the plunger rod assembly 12 shows the two components 22, 24 press-fit together, alternate means of assembling the device 10, including, but not limited to, snap-fit, pins, adhesives and mating threads, may also be used and are included within the scope of the claimed invention.

As best seen in FIGS. 8 and 10, the elongate body 22 includes a longitudinal groove or cut-out 58 that extends along a portion of the body 22. The cut-out 58 forms an opening surrounded by a distal surface 60, a proximal surface 62 and a bottom surface 64 that extends within the elongate body 22 to a depth approximately one-third of its total diameter. The cut-out 58 provides rotational limits for the elongate body 22 relative to the forward tube 26. In particular, a pin 65 (best seen in FIG. 3) located near the proximal end of the forward tube 26 cooperates with the cut-out 58 of the elongate body 22, thereby preventing rotation of the elongate body 22 relative to the forward tube 26.

Opposite to the cut-out 58 and near the distal end 52 of the elongate body 22 is a relatively shallow cylindrical hole or opening 66. The opening 66 is positioned transverse to the longitudinal axis of the elongate body 22 and extends through the bottom surface 58 of the cut-out 58. As shown in FIG. 3, the opening 66 is sized to accommodate the stem 68 of the latch pin 70.

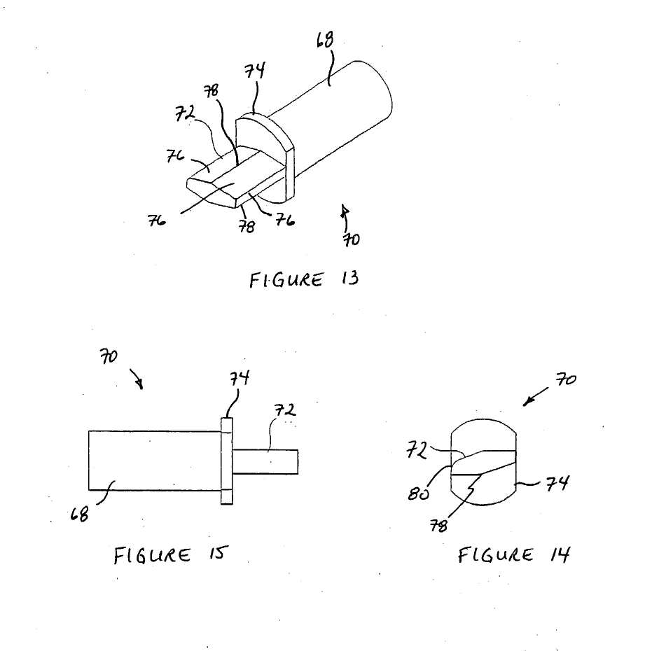

Referring to FIGS. 13-15, one embodiment of the latch pin 70 in accordance with the present invention includes a post 72, a collar 74 and a stem 68. As described above, the stem 68 of the latch pin 70 is configured for engagement with the opening 66 in the elongate body 22. When inserted within the opening 66, the post 72 of the latch pin 70 protrudes from the exterior surface of the elongate body 22. As shown in FIGS. 13 and 14, the post 72 includes a plurality of sides or surfaces 76, with adjacent surfaces forming sharply angled edges 78 there-between. Preferably, one of the post's edges 78 is not angled but, rather, includes a radius of curvature 80. In this configuration, the latch finger of the inserter housing (not shown) is able to easily slide over the curved edge 80 of the latch pin 70 and catch or hook onto one of the angled edges 78 of the post 72 during the IOL insertion procedure, as further explained below. A collar 74, positioned between the post 72 and the stem 68, provides additional support to further stabilize the latch pin 70 in the opening 66 and prevent it from being unintentionally pulled out of the elongate body 22 during use of the device 10.

Although the illustrated embodiment shows the two components 70, 22 press-fit together, alternate means of assembling the latch pin 70 into the elongate body 22, including, but not limited to, snap-fit, pins, adhesives and mating threads, may also be used and are included within the scope of the claimed invention. In another embodiment of the invention, the latch pin 70 and elongate body 22 may be fabricated as a single, continuous component whereby the latch pin 70 is configured, for example, as a projection on the external surface of the elongate body 22 (not shown). Additional component designs and assembly methods, not disclosed herein but known by those skilled in the art, are also included within the scope of the claimed invention.

As shown in FIG. 3, a helical compression spring 82 and slide ring 84 are located on the external surface near the proximate end of the plunger rod assembly 22. The slide ring 84, which is configured as an annular rim or shoulder against which the proximate end of the spring 82 acts, is moveable along a proximate portion of the plunger rod assembly 22. The spring 82, which is also moveable along a proximate portion of the plunger rod assembly 22, acts between the proximate end of the forward tube 26 and the slide ring 84, thereby forcing the slide ring 84 rearward (proximally) along the plunger rod assembly 22. However, rearward travel of the slide ring 84 is limited by engagement against an inturned lip and/or one or more pins 86 near the proximate end of the rearward tube 28. As explained in further detail below, the particular configuration of the slide ring 84 and compression spring 82 limits movement of the plunger rod assembly 12 so that a user of the device can controllably deliver an IOL into the eye of a patient.

Referring back to FIGS. 8-10, the proximal end of the elongate body 22 of the plunger rod assembly 12 further includes an annular channel or groove 88. The annular groove 88 is sized to accommodate one or more pins 90 which project from an interior surface of the control knob assembly 20 (not shown) and extend into the annular groove 88 of the plunger rod assembly 12. As explained in further detail below, moving the control knob assembly 20 along the longitudinal axis of the device results in equivalent longitudinal movement of the plunger rod assembly 12. However, rotating the control knob assembly 20 about the axis of the device does not produce corresponding rotation of the plunger rod assembly 12, as the pins 90 of the control knob assembly 20 can freely rotate within the groove 88 and about the axis of the plunger rod assembly 12. From this description, it is evident that accurate and reliable positioning of the IOL into the eye of the patient are controlled, in part, by the control knob assembly 20 of the present invention. Hence, additional disclosure concerning the structure and function of this assembly is helpful to further understand the present invention. For this purpose, attention is directed to FIGS. 16-18.

control Knob Assembly

FIGS. 16-18 depict one embodiment of the control knob assembly 20 of the inserter device 10 which includes a cylindrically shaped, hollow stem 92 in communication with a disc-shaped handle 94. The disc-shaped handle 94 further includes a circular pushing surface 96 and a ring-shaped gripping surface 98. The pushing surface 96 and ring-shaped gripping surface 98 are configured for a user's finger(s) to securely press against the surface and rotate the knob assembly 20 during an injection procedure.

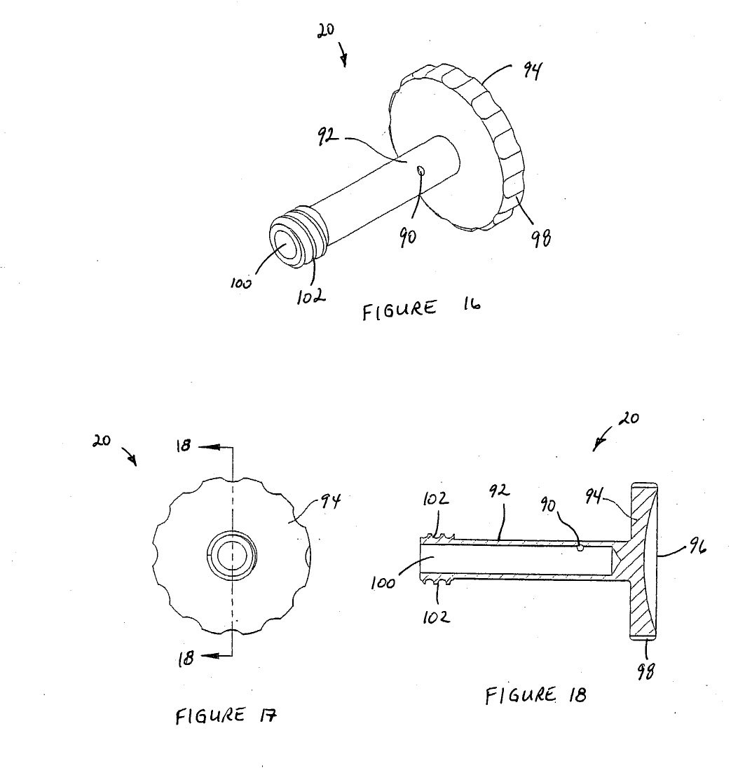

Adjacent the disc-shaped handle 94 is the stem 92 of the control knob assembly 20. As shown in FIGS. 16 and 18, the bore or internal diameter 100 of the hollow stem 92 is sized to accommodate the proximal end of the plunger rod assembly 12 (not shown). Located on the interior surface and near the proximal end of the stem 92 are one or more pins 90. The pins 90 are configured to project inwardly from the stem's interior surface and extend into the annular groove 88 of the plunger rod assembly 12 when assembled therein. As such, the control knob assembly 20 is free to rotate relative to the plunger rod assembly 12 without corresponding rotation of the plunger rod assembly 12.

The distal end of the control knob assembly 20 includes one or more threads 102 formed on the stem's exterior surface. The threaded section 102 of the stem 92 is configured for engagement with one or more inwardly projecting pins 86 formed on the interior surface near the proximal end of the rearward tube 28 of the inserter housing (not shown). As such, when the control knob assembly 20 is moved laterally within the rearward tube 28 of the inserter housing 14, the pin(s) 86 of the rearward tube 28 abut the distal-most thread 102 of the stem 92 and prevent further lateral movement of the knob assembly 20. To laterally advance the control knob assembly 20, the knob assembly 20 must be rotated about its axis. Rotation of the knob assembly 20 causes the pin(s) 86 of the inserter housing 14 to run along the groove of the external threads 102 of the stem 92 resulting in lateral movement of the knob assembly 20.

Inserter Housing

Referring back to FIG. 3, the inserter housing 12, which includes a forward tube 26 and a rearward tube 28, houses the plunger rod assembly 12 and includes a chamber 30 configured to hold the cartridge 32 during an insertion procedure. As described above, the rearward tube 28 includes one or more pins 86 near its proximal end which cooperate with external threads 102 on the control knob assembly 20 to control lateral movement of the control knob 20 and plunger rod assemblies 12. In addition, the rearward tube 28 also includes an annular rib or shoulder 104 located on its exterior surface. The annular rib 104 together with the distal end of the protective sheath 16 secure the finger grip 18 to the device 10 for added positioning control. Although the illustrated embodiment of the device 10 shows the two components press-fit together, alternate means of assembling the device 10, including, but not limited to, snap-fit, pins, adhesives and mating threads, in addition to forming the finger grip 18 as an integral member of the rearward tube 28 or protective sheath 16, may also be used and are included within the scope of the claimed invention.

Located near the distal end of the rearward tube 28 is an attachment section which is used to attach and/or secure the forward tube thereto. As depicted in FIG. 3, the attachment section includes an internally threaded portion 106 configured for engagement with an externally threaded portion 108 on the proximal end of the forward tube 26. Although the illustrated embodiment of the device 10 shows the two components 26, 28 threaded together, alternate means of assembling the components 26, 28, including, but not limited to, snap-fit, pins, and adhesives, may also be used and are included within the scope of the claimed invention.

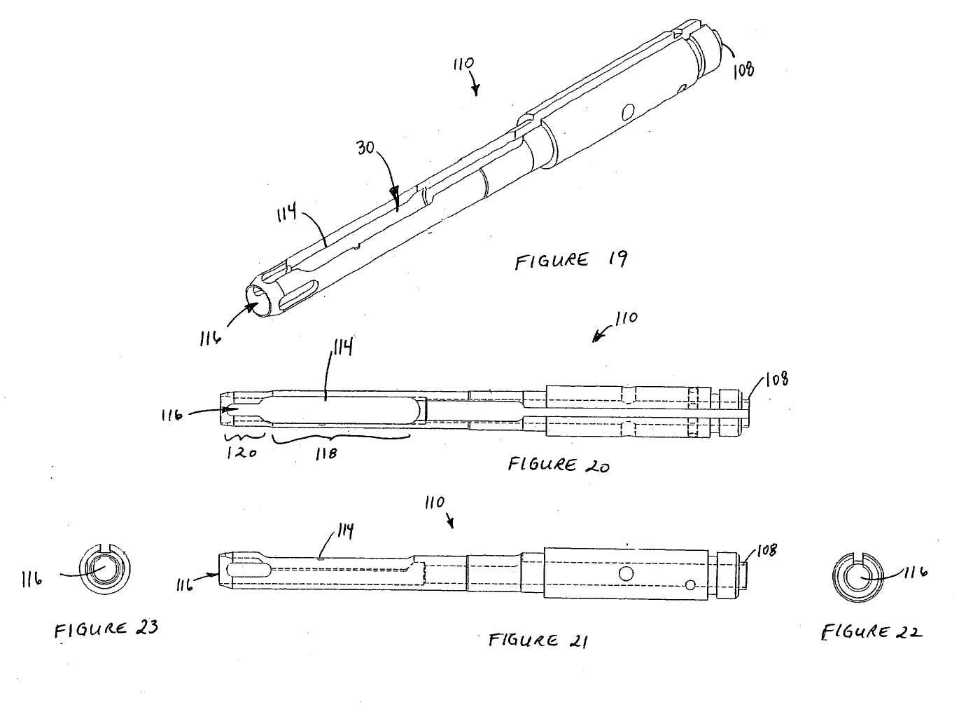

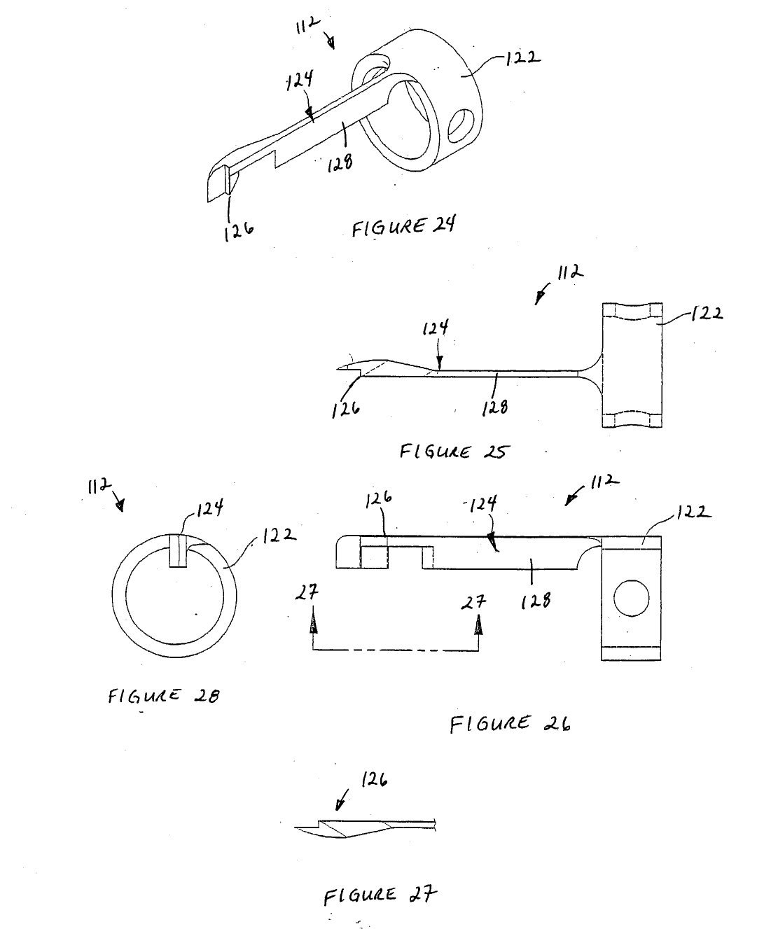

Referring to FIGS. 19-28, the forward tube 26 of the inserter housing 12 includes a body member 110 and clip 112. A slot 114, formed on the external surface and in communication with the internal bore 116 of the body member 110, extends along the longitudinal axis of the body member 110 and is configured to allow the loaded lens holding cartridge 32 to be inserted therein. In particular, the wider portion 118 of the slot 114 allows the loaded cartridge 32 to be inserted downward into the interior bore 116 of the body member 110 and slid forward (distally) to the position shown in the FIG. 1. The narrow, distal portion 120 of the slot 114 stabilizes the cartridge 32 and holds the tabs 42 in a closed position.

As shown in FIGS. 3 and 24-28, the clip 112 of the forward tube 26 includes a mounting ring 122 in communication with a latch finger 124. The mounting ring 122, which attaches to the mid-section of the forward tube 26, is axially aligned on the forward tube 26 so that the latch finger 124 extends over and partially within the slot 30, proximal to the wider portion 114 of the slot 30. The latch finger 124, which includes an angled tip 126 at the distal end of the cantilevered arm 128, is configured to catch or hook onto the latch pin 70 of the plunger rod assembly 12 as the plunger rod assembly 12 is moved toward the distal end of the forward tube 26. Engagement of the latch finger 124 with the latch pin 70 prevents retraction or proximal movement of the plunger rod assembly 12 thereby enabling a user of the device 10 to controllably release the IOL into the chamber of the eye, as described in further detail below.

Operation

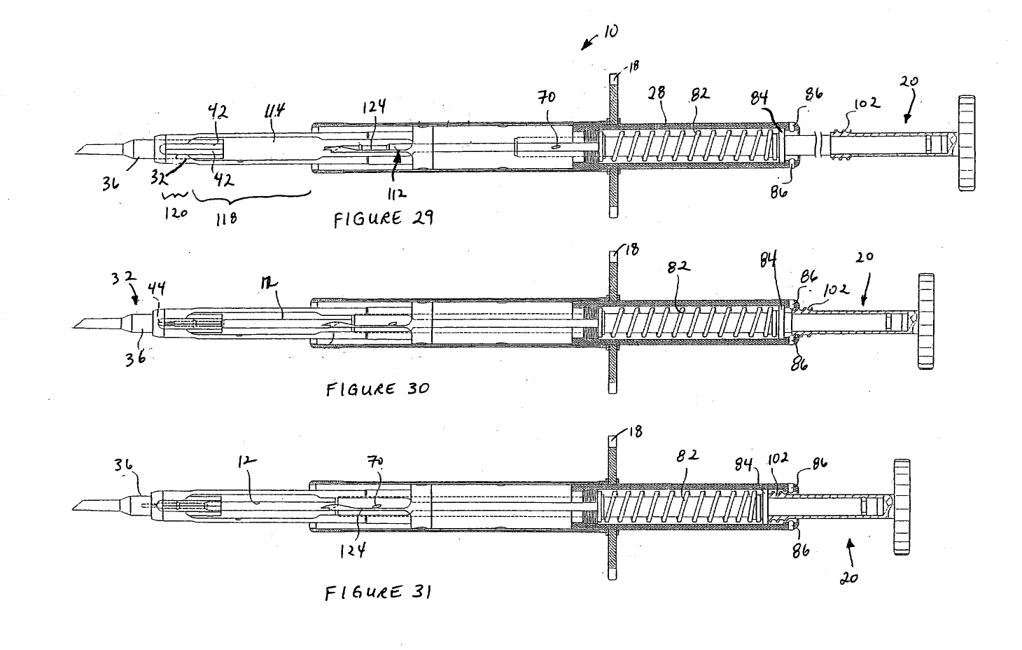

The device 10 in accordance with the present invention is prepared for use by first fitting an IOL (not shown) in the lens-holding cartridge 32 and folding or rolling the IOL by manipulation of the cartridge tabs 42. Next, the cartridge 32 is inserted downward through the wider portion 118 of the slot 114 and forward (distal) such that the tabs 42 fit in the narrower portion 120 of the slot 114. During this procedure, as shown in FIG. 29, the push rod assembly 112 is retracted rearward (proximal) from the wider portion 118 of the slot 114. Note that the latch pin 70 is positioned far to the rear (proximally) of the latch finger 124 and the external threads 102 of the control knob assembly 20 are disengaged from the pins 86 of the rearward tube 28. In addition, the compression spring 82 has forced the slide 82 rearward (proximally) adjacent to the proximate end of the rearward tube 28.

With reference to FIG. 30, the control knob assembly 20 is then slid forward (distally) resulting in equivalent distal movement of the plunger rod assembly 12. The threads 102 of the control knob assembly 20 butt against the pins 86 of the rearward tube 28 at a position in which the distal end 42 of the plunger rod assembly 12 is approximately aligned with the distal end of the lens-folding tabs 42, i.e., the lens is forced from between the tabs 42 into the holding chamber 36. In this configuration, the pins 86 block additional linear sliding movement of the plunger rod assembly 12 in the device 10.

To further advance the plunger rod assembly 12, it is necessary to rotate the control knob assembly 20. In this regard, the external threads 102 cooperate with the pins 86 such that the knob assembly 20 may be turned in a direction to gradually advance the plunger rod assembly 12 from the position shown in FIG. 30 to the position shown in FIG. 31. At the same time, the distal end of the control knob assembly 20 engages the slide 84 and moves the slide 84 distally against the force of the compression spring 82. The axial extent of the threads 102 is configured to move the distal end of the plunger rod assembly 12 approximately half-way into the holding chamber 36 of the cartridge 32. Once the proximate end of the externally threaded portion 102 clears the pins 86, no additional advancement of the plunger rod assembly 12 occurs with continued rotation of the control knob assembly 20. In addition, an audible “click” will sound when the proximal-most thread 102 clears the pins 86 and the slide 84 and control knob assembly 20 are biased rearwardly (proximally) by the compression spring 82.

In the position shown in FIG. 31, the latch pin 70 is positioned generally alongside the center portion of the latch finger 124. Further advancement of the plunger rod assembly 12 and latch pin 70 are achieved by pressing the control knob assembly 20 toward the distal end of the device 10, such as by engagement of the handle 94 with the thumb or palm while the fingers (e.g., index and middle fingers) grasp the finger grip 18. As such, any rotational movement of the finger grip 18 and/or protective sheath 16 may be accomplished independent of longitudinal movement of the pushing member 24 and rotational movement of the control knob assembly 20. This configuration allows for increased control and manipulation of the device using a single-handed approach, thereby freeing the user's second hand for other manipulation. Alternatively, a two-handed approach may also be used whereby the user's second hand is used to rotate the protective sheath, independent of longitudinal movement of the pushing member 24.

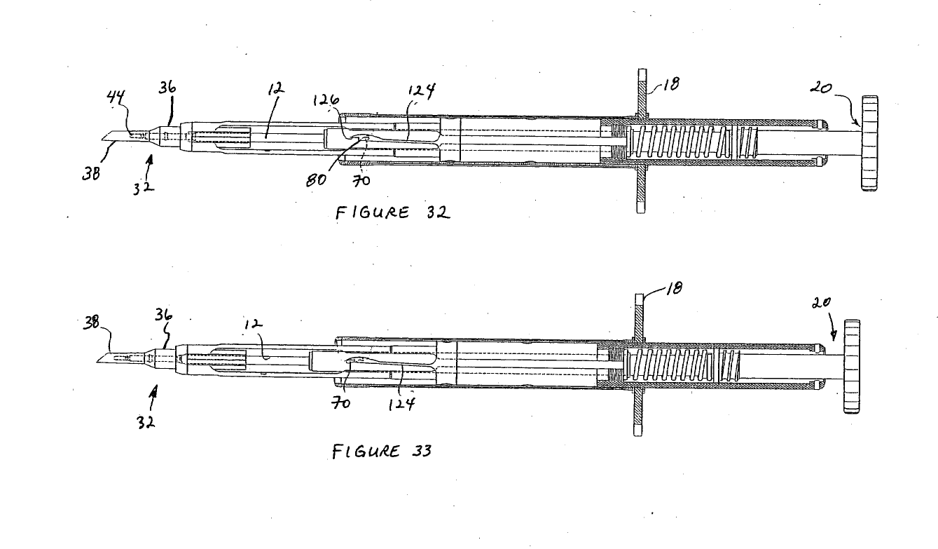

Referring to FIG. 32, the tip 44 and/or distal end of the plunger rod assembly 12 forces the IOL from the holding chamber 36 and into the ejection tube 38. As this occurs, the leading edge 80 of the latch pin 70 engages the angled tip 126 of the latch finger 124, wedging the latch finger 124 sideways and upward in orientation, as shown in FIG. 32. The upward movement of the latch finger 124 is contrary to its natural, centered position. Once the latch pin 70 has cleared the angled tip 126 of the latch finger 124, the latch finger 124 springs back toward its central, relaxed position. When the latch finger 124 is engaged with the latch pin 70, the inward (distal) directed force on the control knob assembly 20 applied by the user's hand is relieved thereby preventing retraction of the plunger rod assembly 12, as shown in FIG. 33. The user of the device can then use the distal end of the ejection tube 38 as a manipulation tool inside the chamber of the patient's eye.

At this point in the procedure, the plunger rod assembly 12 is maintained in a position in which the IOL (not shown) is largely projecting from the distal end of the ejection tube 38, but has not yet been fully pushed out of the cartridge 32. This position allows the user of the device 10 to precisely place the IOL without having to maintain pressure on the control knob assembly 20. When the desired position within the eye has been reached, the knob assembly 20 is further depressed to eject the IOL from the cartridge 32. At the same time, the latch pin 70 moves distally beyond the distal end of the latch finger 124, causing the latch finger 124 to spring back to its relaxed condition. As the force on the control knob assembly 20 is relieved, the latch pin 70 slides rearward along and past the latch finger 124 causing the device 10 to return to the position shown in FIG. 31. The ejection tube is then removed from the corneal incision and the parts returned to their position as shown in FIG. 29 for removal of the empty cartridge 32. At this point, -the device is ready for insertion of a new loaded cartridge.

In addition to the above-described device configurations and method of use, the present invention contemplates alternative embodiments and applications that are also included within the scope of the claimed invention. For example, alternative latch designs may be used in place of or in combination with the latch pin and latch finger designs.

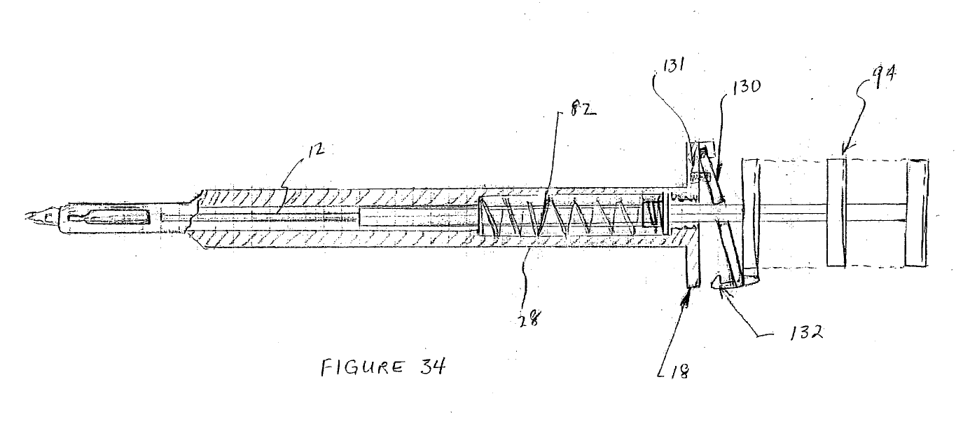

In one embodiment, shown in FIG. 34, an off-axis clamp 130 and lock 132 are located near the proximal section of the rearward tube 28. The off-axis clamp 130 is spring loaded 131 and sized to prevent lateral sliding of the handle 94 in a proximal direction under action of the compression spring 82. This configuration allows the plunger rod assembly 12 to advance the IOL through the cartridge 32 (not shown) without backlash or spring-back of the handle 94. To unlock or release the handle 94, the off-axis clamp 130 is moved in a distal direction toward the finger grip 18. To completely disengage or unlock the handle 94, the lock 132 is latched onto the finger grip 18 of the insertion device 10. The off-axis clamp can be re-engaged with the handle 94 any time throughout the plunger rod translation by simply unlatching the lock 132 from the finger grip 18. This configuration allows a user to freely latch and unlatch the plunger rod assembly 12 throughout operation of the device 10.

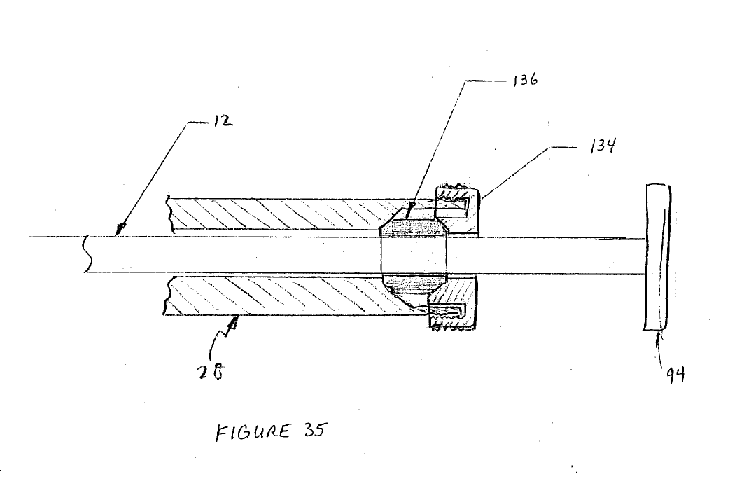

In an alternate embodiment, a lock nut 134 and friction collar 136 are used to prevent lateral movement of the plunger rod assembly 12. As shown in FIG. 35, the lock nut 135 is threaded onto the rearward tube 28 and configured to engage the external surface of a friction collar 136. As the lock nut 134 is secured onto the rearward tube 28, the friction enhanced internal surface of the collar 136 engages the plunger rod assembly 12. Further tightening of the lock nut 134 produces increased friction between the friction collar 136 and plunger rod assembly 12, thereby preventing any lateral movement of the plunger rod assembly 12. To release the locking mechanism, the lock nut 134 is unscrewed or loosened, thereby reducing the amount of friction between the plunger rod assembly 12 and friction collar 136 so that the plunger rod assembly can freely move in a lateral direction.

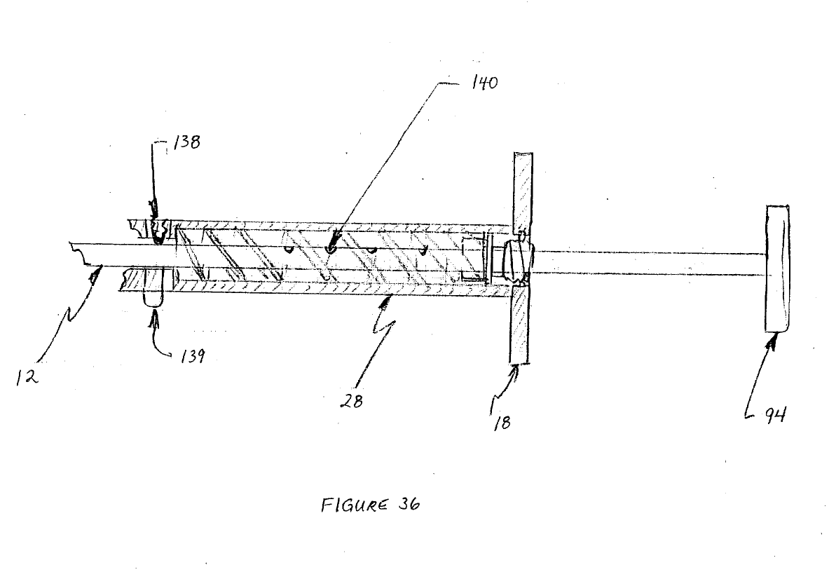

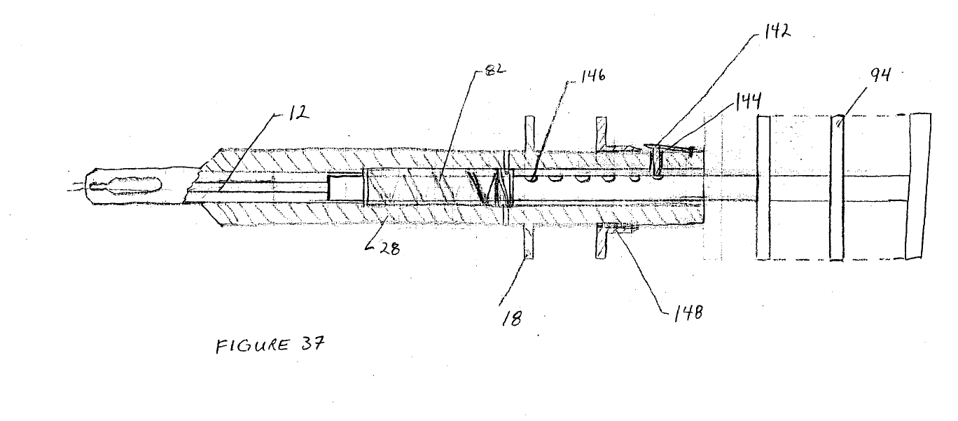

FIG. 36 illustrates an alternate embodiment of the locking mechanism of the present invention. As shown in the illustration, the rearward tube 28 includes a spring plunger 138 and release pin 139. The spring plunger 138 is configured to engage one or more detents 140 located in the handle 94 or plunger rod assembly 12 of the insertion device 10. The lateral position of the plunger rod assembly 12 is locked in place when the spring plunger 138 engages one of the detents 140. Activating the release pin 139 causes the spring plunger 138 to disengage from the detent 140. When the spring plunger 138 is unlocked or disengaged from the detent 140, the plunger rod assembly 12 can once again freely move in a distal or proximal lateral direction.

In an alternate embodiment of the invention, a cantilever spring 142 and pin 144 are attached to the rearward tube 28 and configured to engage one or more detents 146 located on the handle 94. As such, when the plunger rod assembly 12 is advanced, the cantilever spring 142 and pin 144 engage the detents 146 and prevent proximal lateral movement of the plunger rod assembly 12. To release the plunger rod assembly 12 from this locked position, a release collar 148 is pulled in a proximal direction until it engages the cantilever spring 142 and pin 144. This in turn disengages the cantilever spring 142 and pin 144 from the detents 146 to once again allow lateral movement of the plunger rod assembly 12.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A device for inserting, controllably releasing and accurately positioning a folded intraocular lens into an eye comprising:
a plunger rod assembly in communication with an inserter housing, said inserter housing adapted to house an intraocular lens;
a first drive mechanism for providing contact between and causing lateral movement of said plunger rod assembly and said intraocular lens within said inserter housing; and
a positioning mechanism for preventing proximal lateral movement of said plunger rod assembly so that said intraocular lens may be accurately positioned within said eye.

2. A method of inserting an intraocular lens into an eye comprising:
providing an insertion device comprising a plunger rod assembly, an inserter housing and a control knob assembly;
providing a cartridge having a folded intraocular lens positioned therein;
loading said cartridge into said inserter housing;
actuating said control knob assembly to couple a distal end of said plunger rod assembly with said intraocular lens;
inserting a distal end of said cartridge into said eye;
applying a force to said control knob assembly to move said intraocular lens through said cartridge and partially eject said intraocular lens from said cartridge;
actuating a latch and pin mechanism of said plunger rod assembly and removing said force to said control knob assembly without causing movement of said plunger rod assembly;
positioning said intraocular lens in said eye;
applying a force to said control knob assembly to eject said intraocular lens into said eye; and
removing said distal end of said cartridge from said eye.

3. An intraocular lens insertion device comprising:
an inserter housing comprising a plunger rod assembly and a control knob assembly, said control knob assembly having an unlocked position and a locked position;
a cartridge for housing an intraocular lens, said cartridge adapted to be received within said inserter housing and engageable with said plunger rod assembly;
a drive mechanism in communication with said control knob assembly and said plunger rod assembly and capable of causing lateral movement of said plunger rod assembly via said control knob assembly;
a biasing mechanism in communication with said plunger rod assembly to limit the lateral movement of said plunger rod assembly;
a locking mechanism in communication with said control knob assembly for preventing lateral movement of said plunger rod assembly when said control knob assembly is in said locked position.
